(19) United States
(12) Patent Application Publication
(10) Pub. No.: US 2019/0112621 A1
(43) Pub. Date: Apr. 18, 2019

(54) METHOD AND MICROBES FOR THE PRODUCTION OF CHIRAL COMPOUNDS

(71) Applicant: Chain Biotechnology Limited, Oxford (GB)

(72) Inventors: Edward GREEN, London (GB); Dana HELDT, London (GB); Benjamin BRADLEY, London (GB)

(21) Appl. No.: 16/082,505

(22) PCT Filed: Mar. 7, 2017
(86) PCT No.: PCT/GB2017/050601

§ 371 Date: Sep. 5, 2018

(30) Foreign Application Priority Data
Mar. 7, 2016 (GB) 1603915.8

(51) Int. Cl. C12P 7/52; C12P 7/16; C12N 9/04; C12N 9/16

(52) U.S. Cl. CPC C12P 7/52; C12P 7/16; C12N 9/16; C12N 9/0006; C12Y 301/02; C12Y 101/01157

(57) ABSTRACT

The present invention provides a Clostridium species comprising a non-native gene capable of expressing (R)-3-hydroxybutyryl-Co A dehydrogenase. Also provided is a method of producing (R)-3-hydroxybutyric acid, or a salt thereof, and/or (R) 1,3-butanediol using such Clostridium.

The present invention relates to a Clostridium species comprising a non-native gene capable of expressing (R)-3-hydroxybutyryl-CoA dehydrogenase as well as a method of producing (R)-3-hydroxybutyric acid, or a salt thereof, and/or (R)-1,3-butanediol using such Clostridium.

Bio-manufacturing of chemicals using microbial fermentation plays a significant role in the global bioeconomy and is poised for rapid growth over the next decade. The desire for renewable chemicals is driven by a need for cheaper, cleaner and more sustainable products. Of particular importance to the speciality chemical and pharmaceutical industry is the production of functional chiral chemicals with specific stereochemistry.

Chiral chemicals exist in two forms (enantiomers), named R and S which are atomically identical but are non-superimposable mirror images with different arrangements of their constitutive elements. In many (in-vivo) applications one enantiomer of a molecule is inactive and in some cases can be toxic to cells; this is of particular importance to the pharmaceutical and functional food industries where chiral purity is often essential. Chemical synthesis is not specific, thus resulting in mixed chiral enantiomers. Separation to resolve pure enantiomers is expensive and often involves harsh reaction conditions that generate unwanted by-products.

Fermentation process technologies rely on conventional microbes, either yeast, Bacillus or Escherichia coli, that are relatively easy to genetically manipulate and cultivate under aerobic conditions. However, to date, few chemical products have been commercialised, hampered by process inefficiencies. Expanding the palette of domesticated microbial platforms for bio-manufacturing is seen as critical to increase the repertoire of feedstocks and chemical products.

Clostridium bacteria are robust industrial hosts and the Clostridium fermentation process has been used for the commercial production of solvents, acetone and butanol, albeit with mixed success, for almost 100 years. However the metabolic biochemistry is complex and fermentation improvements via strain development have proved to be problematical. Several mutations within the central metabolic pathways involved in butanol production, i.e. the gene encoding crotonase, have been found to be lethal. These factors present a barrier for developing new Clostridium strains for chemical production.

Therefore the preferred approach has been to transfer several genes encoding reductive enzymes from Clostridium (and other difficult to manipulate microbes) into new hosts such as E. coli that are easier to genetically manipulate and cultivate under aerobic conditions. However, this does require complex genetic engineering involving multiple genes.

Tseng et al. (2009) Appl. Environ Microbiol 75(10) p3137-3145 describe a method to produce (R) and (S) 3-hydroxybutyrate in engineered E. coli in aerobic fermentation with glucose. Tseng et al. describes a method to produce both enantiomers from acetyl-CoA by introducing three or five heterologous genes.

Kataoka et al. (2013) Journal of Bioscience and Bioengineering vol. 115(5) p475-480 describe a method to produce (R)-1,3-butanediol using engineered E. coli and aerobic fermentation using glucose. Kataoka et al. describe a method to produce (R)-1,3 butanediol from acetyl-CoA using four heterologous genes.

The present invention addresses the difficulties in the art.

The invention relates to genetically engineered Clostridium bacteria and a fermentation process for the production of chiral chemicals using Clostridium.

The present invention provides a way to genetically engineer Clostridium to produce new chemical products; 3-hydroxybutyric acid, or a salt thereof and/or 1,3-butanediol. More specifically, the invention relates to a method to re-direct carbon flux without requiring gene knockout.

The Clostridium host naturally produces the intracellular metabolite, 3-hydroxybutyryl-CoA from acetoacetyl-CoA in the S-enantiomeric form in a reaction catalysed by 3-hydroxybutyryl-CoA dehydrogenase (Hbd). Subsequently, the (S)-3-hydroxybutyryl CoA is catalysed by crotonase, the second step in the C4 pathway leading to butyric acid and/or butanol production.

The invention relates to the introduction and expression of an (R)-specific hydroxybutyryl-CoA dehydrogenase to produce (R)-3-hydroxybutyryl-CoA rather than the natural S-form. The native crotonase enzyme has a greater specificity for the S-form and cannot efficiently catalyse the R-form. The change in stereochemistry of this intracellular metabolite results in the re-direction of carbon flux down other pathways, that can utilise the substrate, and away from normal C4 pathway leading to butyric acid and butanol.

Subsequently, the introduction of (R)-3-hydroxybutyryl-CoA dehydrogenase produces (R)-3-hydroxybutyryl-CoA (from acetoacetyl-CoA) enabling the production of (R)-3-hydroxybutyrate/(R)-3-hydroxybutyric acid and/or (R)-1,3-butanediol. Neither product is normally produced by Clostridium. The new metabolic pathways provide alternative routes to consume excess reducing power and provide energy in the form of ATP.

The invention provides a fermentation process and a Clostridium species whereby the introduction of a heterologous gene results in a novel Clostridium strain which produce chiral compounds. The process and Clostridium may particularly be used to produce compounds (R)-3-hydroxybutyrate/(R)-3-hydroxybutyric acid and/or (R)-1,3-butanediol, either separately or in combination.

A first aspect of the invention relates to a method of producing (R)-3-hydroxybutyric acid and/or a salt thereof, and/or (R)-1,3-butanediol, the method comprising culturing a Clostridium species comprising a non-native gene capable of expressing (R)-3-hydroxybutyryl-CoA dehydrogenase.

The introduction of the heterologous gene capable of expressing (R)-3-hydroxybutyryl-CoA dehydrogenase, results in the production of the (R) form of 3-hydroxybutyryl-CoA. Native reductase enzymes then convert this (R) form to either (R)-3-hydroxybutyrate/(R)-3-hydroxybutyric acid and/or (R)-1,3-butanediol.

Native enzymes, such as phosphotransbutyrylase (Ptb) and butyrate kinase (Buk), convert (R)-3-hydroxybutyryl-CoA into (R)-3-hydroxybutyrate/(R)-3-hydroxybutyric acid via (R)-3-hydroxybutyrate-phosphate. Whilst native aldehyde and alcohol dehydrogenases or a bifunctional aldehyde/alcohol dehydrogenase (i.e. AdhE) convert (R)-3-hydroxybutyryl-CoA into (R)-1,3-butanediol via (R)-3-hydroxybutyraldehyde. These native reductive enzymes catalyse the R-form, despite the S-form of 3-hydroxybutyryl-CoA being naturally present in Clostridium.

The method can further comprises culturing the Clostridium species under anaerobic or microaerophilic conditions.

The method of the of the invention may include the step of isolating the produced (R)-3-hydroxybutyric acid, or the (R)-3-hydroxybutyrate salt, and/or the (R)-1,3-butanediol from the culture medium.

The method may produce the desired end products, as followed:
the (R)-3-hydroxybutyric acid isomer form comprises 100% of the 3-hydroxybutyric acid formed or the 3-hydroxybutyric acid formed comprises 90-100% in the (R)-3-hydroxybutyric acid isomer form and 0-10% in the (S)-3-hydroxybutyric acid isomer form; and/or
the 1,3-butanediol formed is 100% in the (R)-1,3-butanediol isomer form or the 1,3-butanediol formed comprises 90-100% in the (R)-1,3-butanediol isomer form and 0-10% in the (S)-1,3-butanediol isomer form.

The method of the invention may produce the desired end products in a molar ratio of (R)-3-hydroxybutyrate/(R)-3-hydroxybutyric acid to (S)-3-hydroxybutyrate/(S)-3-hydroxybutyric acid of greater than 5:1, greater than 10:1, greater than 50:1, or greater than 100:1. In one embodiment the ratio of (R)-3-hydroxybutyrate/(R)-3-hydroxybutyric acid to (S)-3-hydroxybutyrate/(S)-3-hydroxybutyric acid is in the range of about 100-5:1, 100-50:1, 100-20:1, 50-5:1, 20-5:1, 15-5:1 or of about 15-10:1.

The method may produce the 1,3-butanediol in a molar ratio of (R)-1,3-butanediol to (S)-1,3-butanediol of greater than 5:1, greater than 10:1, greater than 20:1 or greater than 50:1. In one embodiment the ratio of (R) 1,3-butanediol to (S)-1,3-butanediol is about 100-5:1, 50-5:1, 25-5:1, 15-5:1 or of about 25-10:1.

The invention also relates to (R)-3-hydroxybutyric acid, and/or a salt thereof, and/or (R)-1,3-butanediol produced by the any of the methods described.

A second aspect of the invention relates to a Clostridium species comprising a non-native gene capable of expressing (R)-3-hydroxybutyryl-CoA dehydrogenase.

The (R) 3-hydroxybutyryl-CoA dehydrogenase converts acetoacetyl-CoA to (R)-3-hydroxybutyryl-CoA, in the genetically engineered Clostridium.

The Clostridium species of the invention may be a solventogenic or acidogenic Clostridium species. In one embodiment the Clostridium species is a solventogenic species. When a solventogenic species is used the introduction of a single non-native gene capable of expressing of expressing (R)-3-hydroxybutyryl-CoA dehydrogenase results in a Clostridium strain which can produce (R)-3-hydroxybutyrate/(R)-3-hydroxybutyric acid and/or (R)-1,3-butanediol.

Clostridium species according to the present invention include but are not restricted to Clostridium acetobutylicum, C. arbusti, C. aurantibutyricum, C. autoethanogemum, C. butyricum, C. tyrobutyricum, C. beijerinckii, C. carboxidivorans, C. cellulovorans, C. cellulolyticum, C. diolis, C. homopropionicum, C. lijungdahli, C. kluyveri, C. magnum, C. novyi, C. puniceum, C. ragsdalei, C. roseum, C. perfringens, C. saccharobutylicum, C. saccharolyticum, C. saccharobutylacetonicum, C. saccharoperbutylacetonicum, C. thermocellum, C. tetanomorphum, C. thermobutyricum, C. thermosuccinogenes, C. thermopalmarium, C. tyrobutyricum, C. phytofermentens and C. pasteurianum. Preferably the species is C. acetobutylicum or C. butyricum.

Genes capable of expressing (R)-3-hydroxybutyryl-CoA dehydrogenase (EC1.1.1.36) are selected but are not restricted to genes from a group of organisms including Ralstonia eutropha, (Cupriavidus necator), Bacillus sp, Klebsellia sp, Pseudomonas sp, for examples phbB and phaB. Examples of genes can be found in Table 1.

TABLE 1
R-3-Hydroxybutyryl-CoA Dehydrogenase (EC1.1.1.36)

| Accession | EC | | Protein | | |
|---|---|---|---|---|---|
| No. | number | UniProt Entry name | names | Gene names | Organism |
| P14697 | 1.1.1.36 | PHBB_CUPNH | Acetoacetyl- | phbB phaB | Cupriavidus |
| | | | CoA | H16_A1439 | necator (strain |
| | | | reductase | | ATCC 17699/ |
| | | | (EC1.1.1.36) | | H16/DSM 428/ |
| | | | | | Stanier 337) |
| | | | | | (Ralstonia |
| | | | | | eutropha) |
| P50203 | 1.1.1.36 | PHAB_ACISR | Acetoacetyl- | phaB | Acinetobacter |
| | | | CoA | | sp. (strain |
| | | | reductase | | RA3849) |
| | | | (EC1.1.1.36) | | |
| A0A060V147 | 1.1.1.36 | A0A060V147_KLESP | Acetoacetyl- | phbB | Klebsiella sp. |
| | | | CoA | KQQSB11_260496 | |
| | | | reductase | | |
| | | | (EC1.1.1.36) | | |
| C1D6J5 | 1.1.1.36 | C1D6J5_LARHH | PhaB (EC1.1.1.36) | phaB LHK_01110 | Laribacter |
| | | | | | hongkongensis |
| | | | | | (strain HLHK9) |
| F8GXX8 | 1.1.1.36 | F8GXX8_CUPNN | Acetoacetyl- | phbB | Cupriavidus |
| | | | CoA | CNE_BB1p07810 | necator (strain |
| | | | reductase | | ATCC 43291/ |
| | | | PhbB (EC1.1.1.36) | | DSM 13513/ |
| | | | | | N-1) (Ralstonia |
| | | | | | eutropha) |
| F8GP10 | 1.1.1.36 | F8GP10_CUPNN | Acetoacetyl- | phbB | Cupriavidus |
| | | | CoA | CNE_2c01860 | necator (strain |
| | | | reductase | | ATCC 43291/ |
| | | | PhbB (EC1.1.1.36) | | DSM 13513/ |
| | | | | | N-1) (Ralstonia |
| | | | | | eutropha) |
| G0ETI7 | 1.1.1.36 | G0ETI7_CUPNN | Acetoacetyl- | phaB1 | Cupriavidus |
| | | | CoA | CNE_1c14670 | necator (strain |
| | | | reductase | | ATCC 43291/ |
| | | | PhaB (EC1.1.1.36) | | DSM 13513/ |
| | | | | | N-1) (Ralstonia |
| | | | | | eutropha) |
| A9LLG6 | 1.1.1.36 | A9LLG6_9BACI | NADPH | phaB | Bacillus sp. 256 |
| | | | dependent | | |
| | | | aceto-acetyl | | |
| | | | CoA | | |
| | | | reductase | | |
| | | | (EC1.1.1.36) | | |
| A0A0E0VPS5 | 1.1.1.36 | A0A0E0VPS5_STAA5 | Acetoacetyl- | ST398NM01_1282 | Staphylococcus |
| | | | CoA | | aureus subsp. |
| | | | reductase | | aureus 71193 |
| | | | (EC1.1.1.36) | | |
| D5DZ99 | 1.1.1.36 | D5DZ99_BACMQ | Acetoacetyl- | phaB BMQ_1230 | Bacillus |
| | | | CoA | | megaterium |
| | | | reductase | | (strain ATCC |
| | | | (EC1.1.1.36) | | 12872/ |
| | | | | | QMB1551) |
| V6A8L4 | 1.1.1.36 | V6A8L4_PSEAI | Acetoacetyl- | phbB | Pseudomonas |
| | | | CoA | PAMH27_0609 | aeruginosa |
| | | | reductase | | MH27 |
| | | | (EC1.1.1.36) | | |

In one embodiment the (R)-3-hydroxybutyryl-CoA dehydrogenase gene is phaB. Introduction of phaB can result in the production of two new products by the Clostridium, (R)-3-hydroxybutyrate/(R)-3-hydroxybutyric acid and (R)-1,3-butanediol. The sequence of the phaB gene can be codon optimised for Clostridium. The sequence of phaB may comprise the sequence as shown in FIG. 2A (SEQ ID NO:1).

The nucleic acid encoding the non-native (R)-3-hydroxybutyryl-CoA dehydrogenase may comprise a sequence which has at least 60%, 70%, 80%, 90, 95% or 99% sequence identity with the phaB sequence of FIG. 2A (SEQ ID NO:1).

A number of methods are available to determine identity between two sequences. A preferred computer program to determine identity between sequences includes, but is not limited to BLAST (Atschul et al, Journal of Molecular Biology, 215, 403-410, 1990). Preferably the default parameters of the computer programs are used.

The Clostridium species of the invention may also include a non-native gene capable of expressing thioesterase. Thioesterase converts both the (S) and (R) form of 3-hydroxyburtyryl-CoA to the respective (S) and (R) forms of 3-hydroxybutyrate/3-hydroxybutyric acid.

Genes capable of expressing thioesterase, include but are not limited to TesB. Preferably the TesB is from E. coli. The sequence of the TesB gene can be codon optimised for Clostridium. The sequence of TesB may comprise the sequence as shown in FIG. 2B (SEQ ID NO:2).

The nucleic acid encoding TesB may comprise a sequence which has at least 60%, 70%, 80%, 90, 95% or 99% sequence identity with the TesB sequence of FIG. 2B (SEQ ID NO:2).

In one embodiment, the Clostridium species comprises an S-stereospecific crotonase. Since, it is not desirable to utilise the butyric acid/butanol pathway, it is not advantageous to change the stereo-specificity of crotonase or introduce an (R)-specific crotonase. An (R)-specific crotonase would catalyse the (R)-3-hydroxybutyryl-CoA into crotonyl-CoA and reduce the amount of (R)-3-hydroxybutyryl-CoA available for conversion to (R)-3-hydroxybutyrate/(R)-3-hydroxybutyric acid and/or (R)-1,3-butanediol.

A benefit of the native pathway in Clostridium is native enzymes can catalyse aldehyde/alcohol dehydrogenase reactions and/or 3-hydroxybutyrate reductase reactions. Preferably these native genes are retained in the Clostridium. Therefore in one embodiment the Clostridium species comprises genes that encode for one or more native non-substrate specific dehydrogenase and/or reductase enzymes able to convert (R)-3-hydroxybutyryl-CoA to (R)-3-hydroxybutyrate/(R)-3-hydroxybutyric acid and/or to (R)-1,3-butanediol

Native enzymes for example, Ptb and Buk, convert (R)-3-hydroxybutyryl-CoA into (R)-3-hydroxybutyrate. The (R)-1,3-butanediol is produced from (R)-3-hydroxybutyrate-CoA by native enzymes, such as Bld and/or AdhE, performing the respective aldehyde/alcohol dehydrogenase activities.

However in one embodiment the Clostridium species of the invention may also comprise one or more non-native genes encoding reductive enzymes able to convert (R)-3-hydroxybutyryl-CoA to (R)-3-hydroxybutyrate/(R)-3-hydroxybutyric acid, such as Ptb, Buk or TesB.

The Clostridium species of the invention may also include non-native genes encoding enzymes capable of converting (R)-3-hydroxybutyryl-CoA to (R)-1,3-butanediol, i.e. an aldehyde dehydrogenase and alcohol dehydrogenase (equivalently aldehyde reductase) or a gene expressing an bifunctional aldehyde/alcohol dehydrogenase enzyme able to convert (R)-3-hydroxybutyryl-CoA to (R)-1,3-butanediol in one step.

Preferably the Clostridium comprises one or more non-native genes encoding reductive enzymes to convert (R)-3-hydroxybutyryl-CoA to (R)-1,3-butanediol, when the Clostridium species is an acidogenic Clostridium. In one embodiment the non-native gene may be from another Clostridium species.

These genes may come from organisms including but not limited to Bacillus species, E. coli, or from other strains of Clostridium. Examples of genes encoding these enzymes include but are not limited to:

Aldehyde dehydrogenases: ald (from Clostridium beijerinckii), bld (from Clostridium saccharoperbutylacetonicum), puuC aldH b1300 JW1293 (from Escherichia coli), and aldehyde dehydrogenase from Clostridium butyricum, Clostridium autoethanogenum, Clostridium beijerinckii, Clostridium kluyveri, Clostridium ljungdahlii, Clostridium pasteurianum, Clostridium saccharobutylicum, Clostridium saccharoperbutylacetonicum, Clostridium sporogenes.

Alcohol dehydrogenases and aldehyde reductases: bdhA and bdhB (from Clostridium acetobutylicum), bdh1 and bdh2 (from Clostridium kluyveri), bdh (from Clostridium ljungdahlii), adh 1 (from Clostridium saccharobutylicum) bdh (from Clostridium saccharoperbutylacetonicum) and yqhD (from Escherichia coli).

Aldehyde-alcohol dehydrogenase: adhE (from Bacillus subtilis), adhE and adhE2 (from Clostridium acetobutylicum), and aldehyde-alcohol dehydrogenase from Clostridium autoethanogenum, Clostridium beijerinckii, Clostridium kluyveri, Clostridium ljungdahlii, Clostridium pasteurianum, Clostridium saccharobutylicum, Clostridium saccharoperbutylacetonicum, Clostridium sporogenes and Escherichia coli.

The Clostridium species of the invention may be further genetically engineered to increase the production of (R)-3-hydroxybutyrate/(R)-3-hydroxybutyric acid and (R)-1,3-butanediol. The Clostridium species of the invention may be engineered to knock out butanol, acetone, ethanol, butyrate, acetate and/or lactate production in the Clostridium species.

Accordingly, the invention further comprises a Clostridium species having reduced or knocked out expression of a native gene involved in the production of butanol, acetone and ethanol, butyrate, acetate and/or lactate. Preferably expression of native ptb, buk, hbd and/or alcohol/aldehyde dehydrogenases have been reduced or knocked out of the Clostridium species.

In solventogenic Clostridium species, for example C. saccharoperbutylacetonicum, to increase yields and titres of (R)-1,3-butanediol competing pathways, which include but are not limited to the solvent production pathways of butanol, acetone and ethanol and the organic acid pathways for butyrate, acetate and lactate, can be abolished in the Clostridium species. Knocking out native hbd in the Clostridium species will channel carbon flux away from butanol and butyrate production in the direction of 1,3-butanediol. Knocking out ptb or buk (genes involved in the butyrate pathway) will eliminate carbon flux from (R)-3-hydroxybutyryl-CoA and increase yields of (R)-1,3-butanediol.

In solventogenic Clostridium species, to increase (R)-3-hydroxybutyrate/(R)-3-hydroxybutyric acid production competing solvent and organic acid pathways which include but are not limited to butanol, acetone, ethanol, 1,3-butanediol, acetate and lactate can be knocked out. Knocking out alcohol/aldehyde dehydrogenases will eliminate butanol and (R)-1,3-butanediol.

In acidogenic Clostridium species, such as C. butyricum, to increase (R)-3-hydroxybutyrate/(R)-3-hydroxybutyric acid production competing organic acid pathways which include but are not limited to acetate and lactate, can be knocked out. Knocking out hbd will channel carbon flux away from butyrate production in the direction of 3-hydroxybutyrate.

Knocked out expression, and variants thereof, can include the deletion of an entire gene of interest, its coding portion, its non-coding portion, or any segment of the gene, or a mutation therein, which serves to inhibit or prevent expression or otherwise render inoperable the gene of interest. Standard molecular techniques can be used to knock out or reduced the expression of the selected native genes from the Clostridium species.

In addition to knocking out genes involved in the competing pathways, other methods to increase yields and titres of (R)-3-hydroxybutyrate/(R)-3-hydroxybutyric acid and/or (R)-1,3-butanediol can include optimisation of enzyme activity involved in the (R)-3-hydroxybutyrate/(R)-3-hydroxybutyric acid or (R)-1,3-butanediol production. Such as over expression of heterologous and non-heterologous genes, optimized gene expression and directed enzyme evolution. Optimisation of NADPH cofactor availability for the enzymes of the 1,3-butanediol pathway and the (R)-3-hydroxybutyrate pathway can also be used to increase (R)-1,3-butanediol and (R)-3-hydroxybutyrate/(R)-3-hydroxybutyric acid, as expression of NADP kinase increase (R)-3-hydroxybutyrate/(R)-3-hydroxybutyric acid and (R)-1,3-butanediol titres.

The term “non-native gene” refers to a gene that is not in its natural environment, and includes a gene from one species of a microorganism that is introduced into another species of the same genus.

The non-native genes may be codon optimised for Clostridium and/or placed under the control of promoters that enable controllable expression of the gene in the Clostridium . The expression levels of the enzymes can be optimised by controlling gene expression with inducible promoters and/or promoters with different strength. In one embodiment the non-native genes are placed under the control of a native Clostridium promoter, for example a pfdx or thiolase promoter. In another embodiment, genes are placed under the control of an inducible non-native promoter. Other suitable promoters would be known to the person skilled in the art.

The non-native genes can be introduced in the Clostridium strains by standard plasmid transformation techniques known in the art for producing recombinant microorganisms i.e. conjugation or electroporation.

The genes can be cloned and expressed from an expression vector. These include but are not limited to plasmids. The plasmid containing the non-native gene is introduced into the Clostridium by transformation or conjugation. The expression vectors comprises a nucleic acid sequence encoding for the relevant non-native enzyme and can preferably include a promoter or other regulatory sequences which control expression of the nucleic acid.

Non-native genes, including (R)-3-hydroxybutyryl-CoA dehydrogenase, may be integrated into the chromosome of Clostridium using gene integration technology known to persons skilled in the art, for example using technology as described in WO/2009/101400 or WO/2010/084349.

Accordingly, the invention also comprises a method of producing a Clostridium species capable of producing (R)-3-hydroxybutyrate/(R)-3-hydroxybutyric acid and (R)-1,3-butanediol. The method comprising introducing a non-native gene capable of expressing (R)-3-hydroxybutyryl-CoA dehydrogenase into Clostridium species, for example by plasmid transformation or by integration of the gene into a chromosome of the Clostridium species.

The invention can also comprise a recombinant plasmid for transformation and replication or chromosomal integration of Clostridium. The plasmid comprises a nucleic acid sequence encoding an (R)-3-hydroxybutyryl-CoA dehydrogenase. Preferably the nucleic sequence encodes phaB from Cupriavidus necator.

The recombinant plasmid can further comprise a nucleic acid sequence encoding a thioesterase. Preferably the nucleic acid encodes TesB from E. coli.

The invention also comprises a Clostridium species wherein the non-native gene capable of expressing (R)-3-hydroxybutyryl-CoA dehydrogenase is integrated into the chromosome of the Clostridium. Preferably the invention comprises a Clostridium comprising phaB integrated into the chromosome of a Clostridium. Preferably the gene is integrated into the chromosome of C. saccharoperbutylacetonicum.

Integration of the non-native gene capable of expressing (R)-3-hydroxybutyryl-CoA dehydrogenase, i.e. phaB, into the genome of the Clostridium can surprisingly increase the yield of (R)-1,3-butanediol produced as compared to when plasmid transformation is used to the gene into the species. Therefore a method to increase the yield of (R)-1,3-butanediol in a Clostridium species, can comprise introducing the non-native gene capable of expressing (R)-3-hydroxybutyryl-CoA dehydrogenase into a chromosome of the Clostridium species.

The genetic manipulation and culturing of Clostridium can be performed using methods and culture mediums known to a person skilled in the art. Preferably culturing is performed in anaerobic or microaerophilic conditions. Such methods of culturing Clostridium are described in “Clostridia: Biotechnology and Medical Applications”, Eds H. Bahl and P. Dürre, Wiley-VCH Verlag GmbH, 2001, section 3.4 “Growth conditions and nutritional requirements”, and Bergey's Manual of Systematic Bacteriology, Springer-Verlag New York, (2009). Methods for genetic manipulation include but are not limited to ClosTron (WO/2007/148091), Allele-Coupled Exchange (WO/2009/101400), and methods as described in WO/2010/084349 and WO/2013/144653.

Suitable culture media for culturing Clostridium species are known to the skilled person and include but are not limited to Clostridia Broth Media (CBM), Clostridia Growth Media (CGM), Yeast, Tryptone, Glucose Media (YTG), Reinforced Clostridial Media (RCM) and FCM media.

Suitable batch/continuous/semi-continuous culture systems known to the person skilled in the art can be used to grow the microbes. Strains can preferably be grown in batch cultures in the media described.

Suitable anaerobic conditions may be achieved by cultivation in an anaerobic cabinet flushed with anaerobic gases and by placing the growth media in an anaerobic cabinet 24 hours before use.

After cultivation, the final fermentation broth is a mixture consisting of water, residual substrate, salts, side products (e.g. alcohols, organic acids), but also cells and the target product.

The term “hydroxybutyrate” as used herein has its ordinary meaning as known to those skilled in the art and includes hydroxybutyric acid, its salts, and as well as combinations thereof. The form of (R)-3 hydroxybutyrate produced will depend on the culture conditions and medium used. (R)-3 hydroxybutyrate may be produced as a salt under pH neutral conditions. In acidic conditions (<pH 4.4), the dissociated acid (R)-3-hydroxybutyric acid will predominate.

In order to obtain high purity chemical products, the method of the invention includes isolating the desired end products from the culture. This is achieved by methods known to the person skilled in the art. For example, cells can be separated using centrifugation, filtration, or flocculation. Residual salts and acids can be removed using electrodialysis, salting out, or ion exchange chromatography. Excess water in the broth can be reduced by evaporation. Distillation can then be used to recover purified products such as butanediol. Other methods to recover products include vacuum distillation and solvent extraction including ethyl acetate, tributyl phosphate, diethyl ether, n-butanol, dodecanol, and oleyl alcohol. 3-hydroxybutyrate can be precipitated as a salt of the acid and can be removed using ion exchange chromatography.

Chemicals that can be produced biologically using the methods of the invention are (R)-3-hydroxybutyric acid/(R)-3-hydroxybutyrate (and esters derivatives thereof) and (R)-1,3-butanediol. These two products are not produced by native acidogenic or solventogenic Clostridium. In some embodiments (S)-3-hydroxybutyrate/(S)-3-hydroxybutyric acid and/or (S)-1,3-butanediol can also be produced biologically by the Clostridium.

(R)-1,3-butanediol can be used as building blocks for the synthesis of various optically active compounds such as pheromones, fragrances, and insecticides and as a starting material of chiral azetidinone derivatives and key intermediate of penems and carbapenems for industrial synthesis of beta-lactam antibiotics. 3-hydroxybutyrate and ester derivatives are versatile chiral molecules and are used as a building block for the synthesis of optically active fine chemicals, such as vitamins, antibiotics, pheromones, and flavour compounds, for example in the production of the eye drug Dorzolamide.

The invention provides a method that allows economical commercial production of both these chiral molecules, (R)-3-hydroxybutyrate/(R)-3-hydroxybutyric acid and (R)-1,3-butanediol, and therefore opens up further markets for these products. One such product, a nutraceutical requires both (R)-1,3-butanediol and (R)-3-hydroxybutyrate/(R)-3-hydroxybutyric acid as starting materials. However current chemical synthesis methods and other biological approaches are not cost effective enough to allow commercialisation.

Clostridia are anaerobic bacteria with a fermentative metabolism that naturally convert carbohydrates into a variety of reduced fermentation products. The bacteria have unique metabolic pathways and biochemistry for producing three and four carbon (C3/C4) chemicals.

Naturally occurring ‘acidogenic’ Clostridium, such as C. butyricum, produce acetic and butyric acid but no solvents whereas ‘solventogenic’ Clostridium, such as C. acetobutyicum, first produce acids (acetic and butyric) followed by solvents (acetone and butanol) in a two-stage or biphasic fermentation. The acids are re-assimilated during the fermentation to produce acetone and butanol and this is needed to regulate pH and to maintain a redox balance with reduced fermentation products serving to regenerate cofactors required for cell growth. Solvent production is highly regulated and triggered by low pH and/or build-up of organic acids and only occurs in the latter part of the fermentation.

The metabolic pathway in the parental strain is detailed in FIG. 1A. The native gene encoding 3-hydroxybutyryl-CoA dehydrogenase (Hbd) converts acetoacetyl-CoA into (S)-3-hydroxybutyryl-CoA which in turn is converted into crotonoyl-CoA, in a reaction catalysed by crotonase (Crt). Crotonoyl-CoA is converted to butyryl-CoA which in turn is converted to either butyrate or butanol.

The metabolic pathway of a genetically engineered clostridium strain is detailed in FIG. 1B. A heterologous (R)-3-hydroxybutyryl-CoA dehydrogenase (Enzyme A) is introduced that converts acetoacetyl CoA into the (R)-3-hydroxybutyryl-CoA. The (R)-specific 3-hydroxybutyryl-CoA dehydrogenase competes with the native Hbd enzyme for the substrate (acetoacetyl-CoA).The native crotonase (Crt) enzyme has no or only low activity towards the R-form of 3-hydroxybutyryl-CoA, allowing (R)-3-hydroxybutyryl-CoA to be converted to either (R)-1,3-butanediol or (R)-3-hydroxybutyrate via native enzymes. Enzymes Ptb and Buk are specific for the R-form and convert (R)-3-hydroxybutyryl-CoA into (R)-3-hydroxybutyrate via (R)-3-hydroxybutyryl-phosphate whereas aldehyde dehydrogenase (i.e. Bld) and/or bifunctional aldehyde/alcohol dehydrogenases, (i.e. AdhE) convert (R)-3-hydroxybutyryl-CoA to (R)-1,3-butanediol directly or via (R)-3-hydroxybutyraldehyde.

An alternative route to produce (R)-3-hydroxybutyrate/(R)-3-hydroxybutyric acid is via the introduction of a further heterologous gene encoding a thioesterase i.e. TesB from E. coli (Enzyme B). This enzyme converts (S)- and (R)-3-hydroxybutyryl-CoA into (S)- and (R)-3-hydroxybutyrate, respectively.

The present invention is now described with reference to the examples and the following figures:

### FIGURES

FIG. 1 (A) shows the native acid and solvent production metabolic pathways in solventogenic Clostridium.

FIG. 1(B) shows the acid and solvent production metabolic pathways in solventogenic Clostridium after the introduction of a heterologous (R)-3-hydroxybutyryl-CoA dehydrogenase (A) and a heterologous thioesterase (B).

FIGS. 2A-B shows the codon optimised sequence for: the phaB gene from Cupriavidus necator (A) and the TesB gene from E. coli (B).

FIGS. 3 A-B details the plasmid maps for pfdx_phaB in pMTL83151 (A), pMTL83251 (B) and pMTL82151 (C).

FIG. 4 shows the concentration of (R/S)-3-hydroxybutyrate and (R/S)-1,3-butanediol produced in C. acetobutylicum (p83151-pfdx_PhaB).

FIG. 5 shows the concentration of (R/S)-3-hydroxybutyrate and (R/S)-1,3-butanediol produced in C. acetobutylicum (p83251-pfdx_PhaB).

FIG. 6 shows the concentration of (R)-3-hydroxybutyrate and (R)-1,3-butanediol produced in C. saccharoperbutylacetonicum using plasmid transformation (p82151-pfdx_PhaB) in CGM (A) and in FMC (B).

FIG. 7 shows the concentration of (R)-3-hydroxybutyrate produced in C. acetobutylicum (p83251-pfdx_PhaB).

FIG. 8 shows the concentration of (R/S)-3-hydroxybutyrate produced in C. butyricum (p83151-pfdx_PhaB_TesB) and C. butyricum (p83151-pfdx_PhaB).

FIGS. 9A-B shows the concentration of (R)-3-hydroxybutyrate (A) and (R)-1,3-butanediol (B) produced in C. saccharoperbutylacetonicum when the gene is integrated into a chromosome of the bacteria.

FIGS. 10A-B shows the concentration of (R)-3-hydroxybutyrate (A) and (R)-1,3-butanediol (B) produced in C. saccharoperbutylacetonicum when the gene is integrated into a chromosome of the bacteria or introduced by plasmid transformation.

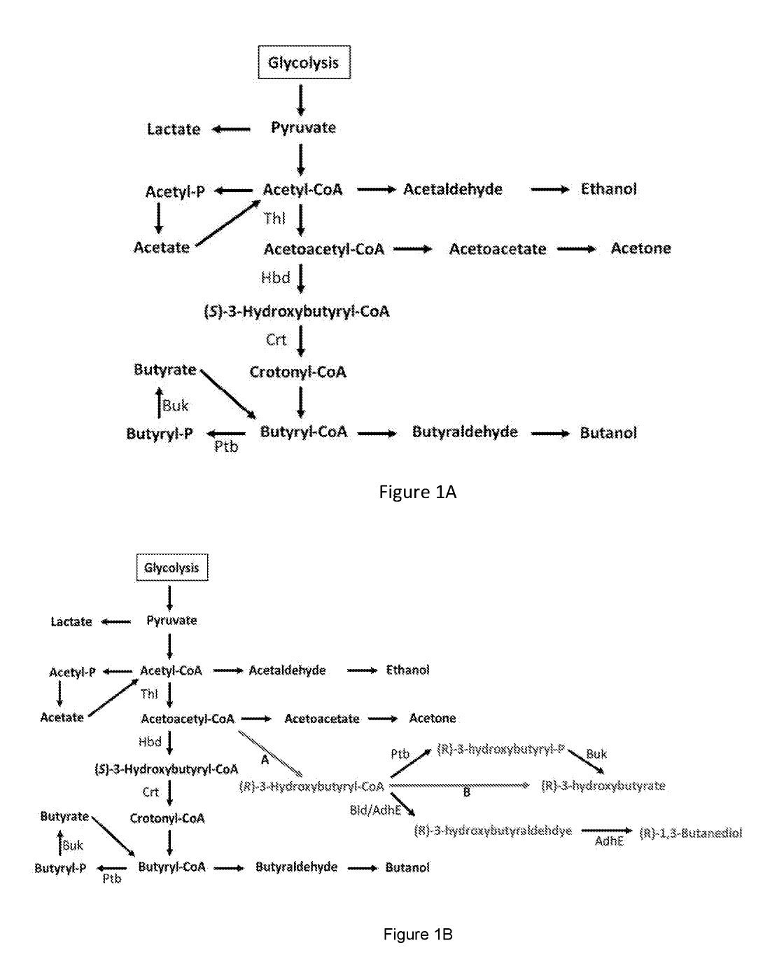

### EXAMPLES

### Example 1

### C. acetobutylicum (PhaB Expression)

### 1) Gene Synthesis

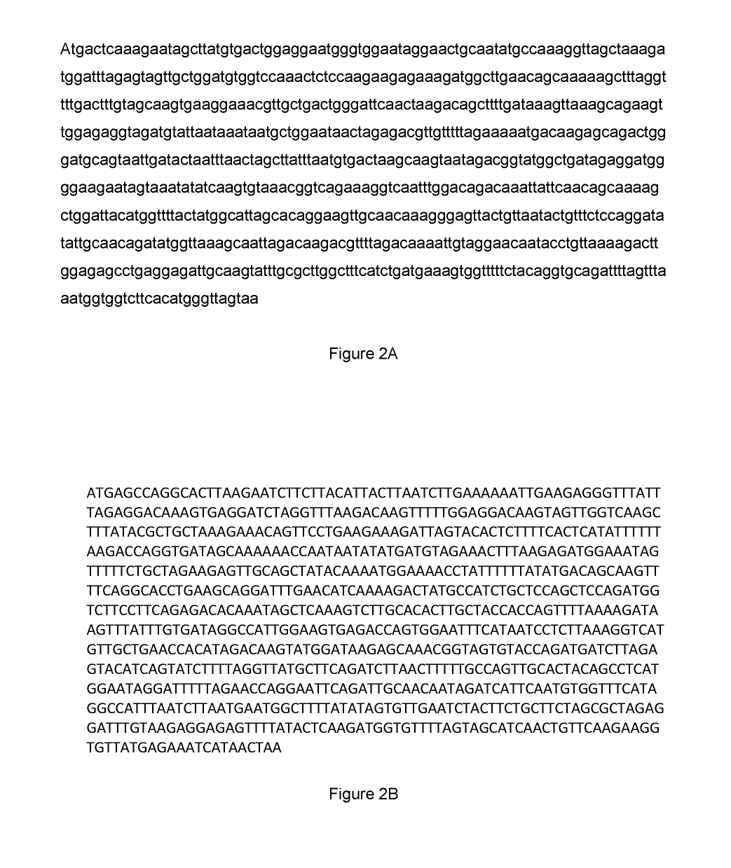

The gene Cupriavidus necator PhaB was codon optimised for Clostridia. FIG. 2A shows one example of the codon optimised sequence which was synthesized by Gene Art® (Thermo Fisher Scientific).

### 2) Plasmid Assembly

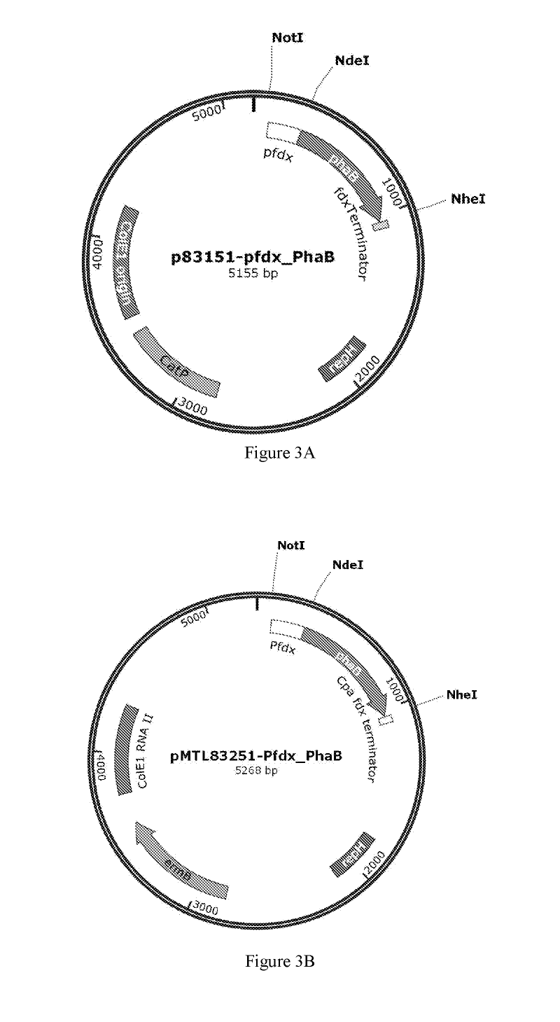

PhaB was cloned into plasmid pMTL83151 using restriction sites NdeI and NheI. The C. sporogenes Pfdx promoter was cloned upstream of the gene using Infusion cloning kit yielding plasmid pMTL83151_pfdx_phaB. PfdX-phaB was extracted from pMTL83151_pfdx_phaB using restriction sites NotI and NheI. The extracted fragment was cloned into pMTL83251 using standard cloning methods (FIGS. 3A and 3B).

### 3) Strain Development

The designed plasmids were used to transform E. coli TOP10 pAN2 for in vitro methylation using standard transformation protocol. The methylated plasmids were extracted using a commercial kit and used to transform C. acetobutylicum ATCC 824. Briefly, an overnight culture of C. acetobutylicum was used to inoculate 2× YTG. Cells were grown anaerobically at 37° C. to an OD₆₀₀ of 0.6-0.8 and were washed with ice cold, anaerobe Electroporation buffer (EPB) (270 mM sucrose, 5 mM sodium phosphate (pH 7.4). The final pellet was re-suspended in a small volume of ice cold, anaerobe EPB and immediately used for transformation. Plasmid DNA (1-2 μg) and cells were added to the pre-chilled 0.4 cm gap cuvette. Electroporation was carried out using a BioRad electroporator with following settings: 2.0 kV, 25 μF and ∞Ω. Transformed cells were recovered anaerobically at 37° C. for 1-3 h in 2× YTG, pH 5.2 before plated on CGM media containing the required antibiotics (15 μg/ml thiamphenicol or 50 μg/ml erythromycin) Single colonies were obtained within 24-48 hours. The presence of the plasmid was confirmed using colony PCR and plasmid specific primers. Transformed colonies were picked for each plasmid and stored as −80° C. freezer stock.

The culture media used:

Suitable culture media include but is not limited to CBM, CGM and 2× YTG media.

Exemplified media are:

CBM containing per 1 L:1 ml FeSO₄×7H₂O (10 mg/mL), 10 ml MgSO₄×7H₂O (20 mg/mL), 1 ml MnSO₄×4H₂O (10 mg/mL), 4 g Casein hydrolysate, 1 ml 4-Aminobenzoic acid (1 mg/ml), 1 ml thiamine-HCL (1 mg/ml), 1.33 μl biotin (1.5 mg/ml), 10 ml K₂HPO₄ (50 mg/ml), 10 ml KH₂PO₄ (50 mg/ml), 20 ml CaCO₃ (250 mg/ml), 2.5-5% glucose. For solid media Agar was added 15 g/L and CaCO₃ omitted.

CGM containing per 1 L:2 g Ammonium Sulphate, 1 g Potassium phosphate dibasic, 0.5 g Potassium phosphate dibasic, 0.2 g Magnesium sulphate heptahydrate, 0.75 ml Iron sulphate heptahydrate (20 g/L), 0.5 ml Calcium Chloride (20 g/L), 0.5 ml Manganese sulphate monohydrate (20 g/L), 0.1 ml Cobalt hydrate ((20 g/L), 0.1 ml Zinc Sulphate (20 g/L), Tryptone 2 g, Yeast extract 1 g, 50 g Glucose, 12 g Agar.

2× YTG containing per 1 L; 16 g tryptone, 10 g yeast extract, 5 g NaCl, pH adjusted to 5.2 and sterilised by autoclaving at 121° C. Sterile glucose is added to cool down media at a concentration of 0.5-2%

4) Fermentation Data for C. acetobutylicum

### Growth Method

Transformed strains were re-streaked from −80° C. freezer stocks on CBM or CGM plates containing the appropriate antibiotics (15 μg/ml thiamphenicol or 50 μg/ml erythromycin) Single colonies were picked and used to inoculate an over-night seed culture (2× YTG, pH 5.2). The seed culture was grown anaerobically at 37° C. for up to 16 h. A 40 ml CGM culture containing 2.5% Glucose was inoculated next day 1:100 using the seed culture. Strains were grown anaerobically at 37° C. Samples for metabolic analysis were taken after 48 hr of growth and analysed for (R/S)-3-hydroxybutyrate (R/S-HB) and (R/S)-1,3-butanediol (R/S-BDO).

### Analysis

Analysis for R/S-3-hydroxybutyrate was carried out using HPLC-MS. The samples were derivatized using DATAN (Diacetyl-tartaric Anhydride) and separation of the S and R form was carried out using a standard non-chiral LC column (Agilent Zorbax Eclipse Plus C18, 2.1×150 mm, 1.8 um). Briefly, 10 μl supernatant was mixed with 250 μl methanol. Samples were dried down at 50° C., followed by the addition of 50 μl of freshly prepared DATAN solution (200 g/l DATAN in dichloromethane: acetic acid 4:1 (v/v). Samples were incubated for 120 min at 75° C., followed by evaporation step. Dried down samples were suspend in 500 μl water and analysed by LC-MS.

### Results

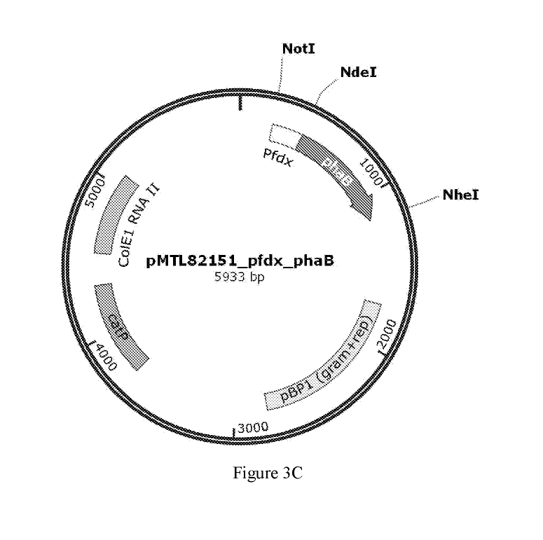

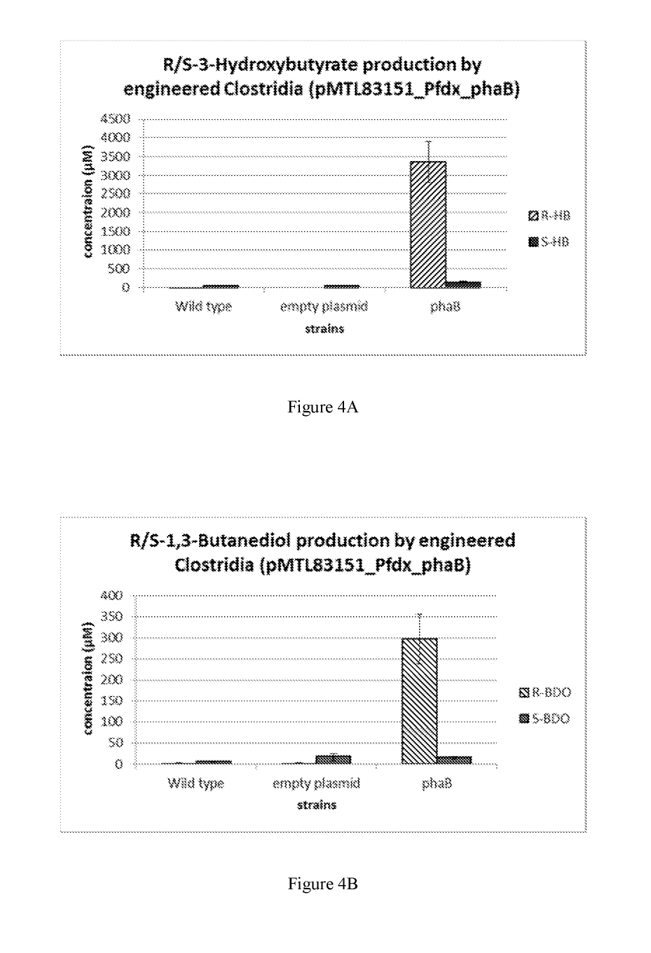

Expression of phaB leads to the production of (R)-3-hydroxybutyrate and (R)-1,3-butaendiol as shown in FIG. 4 (pMTL83151_pfdx_phaB) and FIG. 5 (pMTL83251_pfdx_phaB). Concentrations of over 250 μM of (R)-1,3-butanediol and over 3000 μM of (R)-3-hydroxybutyrate were achieved. None or minimal levels of (S)-1,3-butanediol and (S)-3-hydroxybutyrate were detected in the transformed strains.

### Example 2

### C. saccharoperbutylacetonicum (Plasmid Integration)

### 1) Gene Synthesis

The gene Cupriavidus necator PhaB was codon optimised for Clostridia. FIG. 2A shows one example of the codon optimised sequence which was synthesized by Gene Art® (Thermo Fisher Scientific).

### 2) Plasmid Assembly

PhaB, together with the C. sporogenes Pfdx promoter was cloned into plasmid pMTL82151 using restriction sites NotI and NheI yielding plasmid pMTL82151_pfdx_phaB.

### 3) Strain Development

Plasmid pMTL82151_pfdx_phaB was used to transform Clostridium saccharoperbutylacetonicum (Cspa) by standard electroporation methods. Briefly, cells were grown anaerobically at 37° C. to an OD₆₀₀ of 0.6-0.8 and were washed with ice cold, anaerobe Electroporation buffer (EPB) (270 mM sucrose, 5 mM sodium phosphate (pH 7.4). The final pellet was re-suspended in a small volume of ice cold, anaerobe EPB and immediately used for transformation. Plasmid DNA (1-2 μg) and cells were added to the pre-chilled 0.4 cm gap cuvette. Electroporation was carried out using a BioRad electroporator with following settings: 2.0 kV, 25 μF and ∞Ω. Transformed cells were recovered anaerobically at 37° C. in RCM, pH 5.2 before plated on RCM +50 μg/ml Chloramphenicol. Single colonies were obtained within 24-48 hours. The presence of the plasmid was confirmed using colony PCR and plasmid specific primers. Transformed colonies were picked for each plasmid and stored as −80° C. freezer stock.

4) Fermentation Data for C. saccharoperbutylacetonicum

### Growth Method

Transformants were grown overnight in seed cultures (growth media: CGM or FMC) at 37° C. A 40 ml CGM culture containing 5% glucose was inoculated the next day to a starting OD of 0.05-0.1. Strains were grown anaerobically at 37° C. Samples for metabolic analysis were taken at regular intervals and analysed for (R/S)-3-hydroxybutyrate (R/S-HB) and (R/S)-1,3-butanediol (R/S-1,3-BDO).

### Analysis

Supernatant samples were analysed using a Aminex Ion-Exclusion Column (HPX-87H, 300 mm 7.8 mm, Bio-Rad) connected to an HPLC. Metabolites were eluted with 5 mM H₂SO₄ at a flow rate of 0.5 ml min

Chirality analysis of produced 3-hydroxybutyrate and 1,3-butanediol was carried out using HPLC-MS. The samples were derivatized using DATAN (Diacetyl-tartaric Anhydride) and separation of the S and R forms was carried out using a standard non-chiral LC column (Agilent Zorbax Eclipse Plus C18, 2.1×150 mm, 1.8 um).

Briefly, 10 μl supernatant was mixed with 250 μl methanol. Samples were dried down at 50° C., followed by the addition of 50 μl of freshly prepared DATAN solution (200 g/l DATAN in dichloromethane:acetic acid 4:1 (v/v)). Samples were incubated for 120 min at 75° C., followed by evaporation step. Dried down samples were suspend in 500 μl water and analysed by LC-MS.

### Results

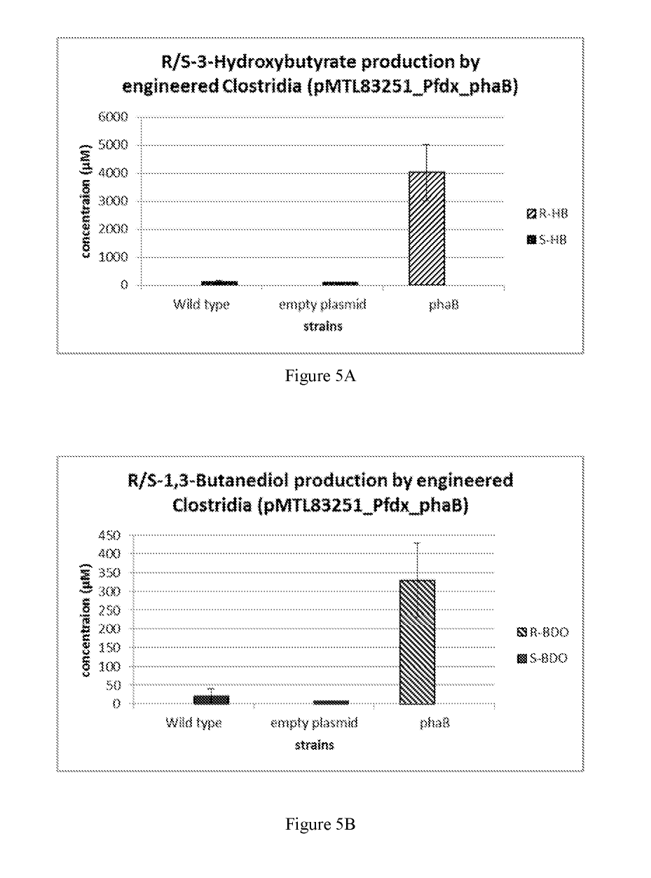

Expression of phaB in Clostridium saccharoperbutylacetonicum results in the production of (R)-3-hydroxybutyrate and (R)-1,3-butanediol, as shown in FIG. 6.

Growth media depending, about 5.5-7 mM 3-hydroxybutyrate and 5-6 mM 1,3-butanediol was produced within 72 h. Mass spec analysis confirmed R-chirality of the produced 3-hydroxybutyrate and 1,3-butanediol.

### Example 3

### C. butyricum

### 1) Gene Synthesis

The gene Cupriavidus necator PhaB was codon optimised for Clostridia. FIG. 2A shows one example of the codon optimised sequence which was synthesized by Gene Art® (Thermo Fisher Scientific).

### 2) Plasmid Assembly

PhaB was cloned into plasmid pMTL83251 under control the C. sporogenes Pfdx promoter using standard cloning techniques yielding plasmid pMTL83251_pfdx_phaB.

### 3) Strain Development

Plasmid pMTL83251_pfdx_phaB was conjugated into Clostridium butyricum using E. coli CA434. A standard conjugation protocol was applied. Briefly, overnight cultures of E. coli CA434 carrying plasmid pMTL83251_pfdx_phaB and C. butyricum were used to inoculate 9 ml LB media and RCM respectively. Cultures were grown until OD of 0.5-0.7. 1 ml of E. coli culture was spun down and the pellet mixed with 200 μl C. butyricum culture. The cell mix was spotted on a non-selective RCM plate and incubated overnight. The incubated mix was re-suspended into 500 μl fresh RCM and plated on selective media containing 10 μg/ml erythromycin. Presence of the plasmid within the obtained transconjugants was confirmed by PCR using plasmid specific primers.

4) Fermentation Data for C. butyricum

### Growth Method

RCM containing per 1 L: yeast extract 13 g, Peptone 10 g, soluble starch 1 g, sodium chloride 5. g, sodium acetate 3 g, cysteine hydrochloride 0.5 g, carbohydrate 2%, was used. Calcium carbonate 10 g/L were added to liquid culture for pH regulation. Solid media contained 15 g/L agar.

Transformants were grown overnight in seed cultures (RCM) at 37° C. 100 ml RC media containing 2% glucose was inoculated to a starting OD of 0.05-0.1. Strains were grown anaerobically at 37° C. in the presence of required antibiotic. Samples for metabolic analysis were taken at regular intervals.

### Analysis and Results

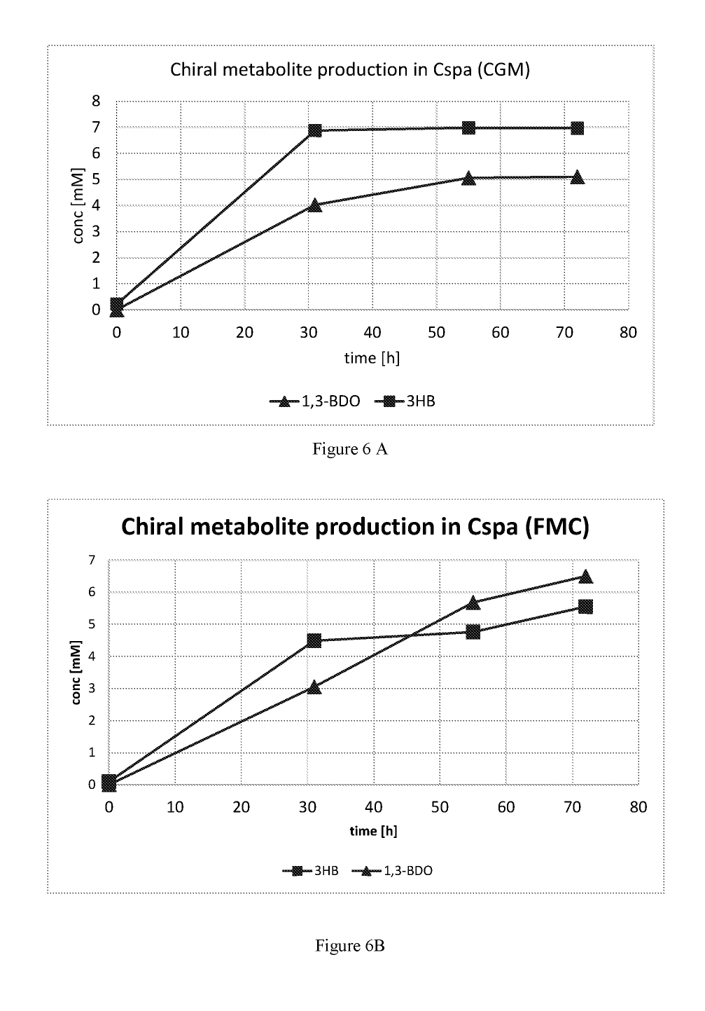

Culture supernatant was analysed for (R)-3-hydroxybutyrate using the 3-hydoxybutyrate assay kit (Sigma Aldrich). The strain expressing phaB produced about 17 mg/L 3-hydroxybutyrate as shown in FIG. 7.

### Example 4

### C. acetobutylicum

### 1) Gene Synthesis

The genes Cupriavidus necator PhaB and E. coli TesB were codon optimised for Clostridia. FIG. 2A shows one example of the codon optimised phaB sequence which was synthesized by Gene Art® (Thermo Fisher Scientific). FIG. 2B shows one example of the codon optimised TesB sequence which was synthesized by Gene Art® (Thermo Fisher Scientific).

### 2) Strain Development

PhaB and TesB were cloned as one operon into pMTL83151 under control of the pfdx promoter using standard cloning techniques. The generated plasmid was used to transform E. coli TOP10 pAN2 for in vitro methylation using standard transformation protocol. The methylated plasmids were extracted using a commercial kit and used to transform C. acetobutylicum ATCC 824. Briefly, an overnight culture of C. acetobutylicum was used to inoculate 2× YTG. Cells were grown anaerobically at 37° C. to an OD₆₀₀ of 0.6-0.8 and washed with ice cold, anaerobe Electroporation buffer (EPB) (270 mM sucrose, 5 mM sodium phosphate (pH 7.4). The final pellet was re-suspended in a small volume of ice cold, anaerobe EPB and immediately used for transformation. Plasmid DNA (1-2 μg) and cells were added to the pre-chilled 0.4 cm gap cuvette. Electroporation was carried out using a BioRad electroporator with following settings: 2.0 kV, 25 μF and ∞Ω. Transformed cells were recovered anaerobically at 37° C. for 1-3 h in 2× YTG, pH 5.2 before plated on CGM media containing 15 μg/ml thiamphenicol. Single colonies were obtained within 24-48 hours. The presence of the plasmid was confirmed by colony PCR using plasmid specific primers. Transformed colonies were picked for each plasmid and stored as −80° C. freezer stock.

3) Fermentation Data for C. acetobutylicum
Growth Method Transformants were grown overnight in seed cultures (growth media: CBM) at 37° C. Samples were taken at regular intervals and production of chiral chemicals (R)-1,3-butanediol and (R)-3-hyrdoxybutyrate analysed by HPLC_MS.

### Analysis

Analysis for R/S-3-Hydroxybutyrate was carried out using HPLC-MS. The samples were derivatized using DATAN (Diacetyl-tartaric Anhydride) and separation of the S and R form was carried out using a standard non-chiral LC column (Agilent Zorbax Eclipse Plus C18, 2.1×150 mm, 1.8 um). Briefly, 10 μl supernatant was mixed with 250 μl methanol. Samples were dried down at 50° C., followed by the addition of 50 μl of freshly prepared DATAN solution (200 g/l DATAN in dichloromethane: acetic acid 4:1 (v/v). Samples were incubated for 120 min at 75° C., followed by evaporation step. Dried down samples were suspend in 500 μl water and analysed by LC-MS.

### Results

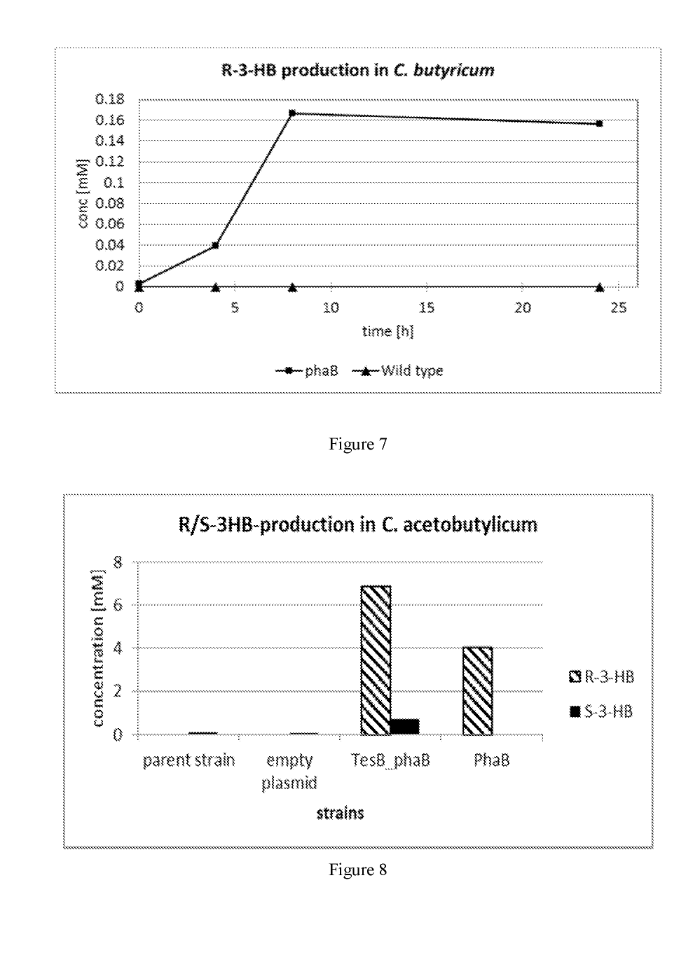

Overexpression of phaB in C. acetobutylicum leads to the production of two chiral chemicals (R)-1,3-butanediol and (R)-3-hyrdoxybutyrate as shown in FIG. 8. Addition of TesB increased the titres of (R)-3-hydroxybutyrate by 1.5×. TesB is non-chiral specific and can use S/R-3-HB-CoA as substrate resulting in a strain producing (R)-3-hydroxybutyrate and (S)-3-hydroxybutyrate at a ratio of about 10:1.

### Example 5

### C. saccharoperbutylacetonicum (Genome Integration)

### 1) Gene Synthesis

The gene Cupriavidus necator PhaB was codon optimised for Clostridia. FIG. 2 shows one example of the codon optimised sequence which was synthesized by Gene Art® (Thermo Fisher Scientific).

### 2) Strain Development

PhaB was integrated into the genome of C. saccharoperbutylacetonicum using the published ACE method based on pyrE (for example as described WO2009/101400). Transformants were confirmed using gene specific primers.

3) Fermentation for C. saccharoperbutylacetonicum

### Growth Method

Transformants were grown overnight as provided below using suitable culture media include but are not limited to FMC and CGM. Exemplified media are:

FMC media containing per 1L: Yeast extract 2.5 g, Tryptone 2.5 g, FeSO₄×7H₂O 0.025 g (NH4)₂SO₄ 0.5 g. The pH was checked and adjust before autoclaving to 6.5-7. CaCO₃ 5 g-10 g was added to regulate the pH.

CGM media containing per 1L (pH 6.6): yeast extract 5 g, NaCl 1 g, K₂HPO₄ 0.75 g, KH₂PO₄ 0.75 g, MgSO₄*7H₂O 0.4 g, FeSO₄*7H₂O 0.01 g, MnSO₄*4H₂O 0.01 g, (NH₄)₂SO₄ 2 g, asparagine 2 g. Calcium carbonate 5-10 g/L was added to liquid culture for pH regulation

Samples for metabolic analysis were taken at regular interval and analysed for (R/S)-3-hydroxybutyrate (R/S-HB) and (R/S)-1,3-butanediol (R/S-1,3-BDO).

### Analysis

Supernatant samples were analysed using a Aminex Ion-Exclusion Column (HPX-87H, 300 mm 7.8 mm, Bio-Rad) connected to an HPLC. Metabolites were eluted with 5 mM H₂SO₄ at a flow rate of 0.5 ml min.

Chirality analysis of produced 3-hydroxybutyrate and 1,3-butanediol was carried out using HPLC-MS. The samples were derivatized using DATAN (Diacetyl-tartaric Anhydride) and separation of the S and R form was carried out using a standard non-chiral LC column (Agilent Zorbax Eclipse Plus C18, 2.1×150 mm, 1.8 um). Briefly, 10 μl supernatant was mixed with 250 μl methanol. Samples were dried down at 50° C., followed by the addition of 50 μl of freshly prepared DATAN solution (200 g/I DATAN in dichloromethane:acetic acid 4:1 (v/v). Samples were incubated for 120 min at 75° C., followed by evaporation step. Dried down samples were suspend in 500 μl water and analysed by LC-MS.

### Results

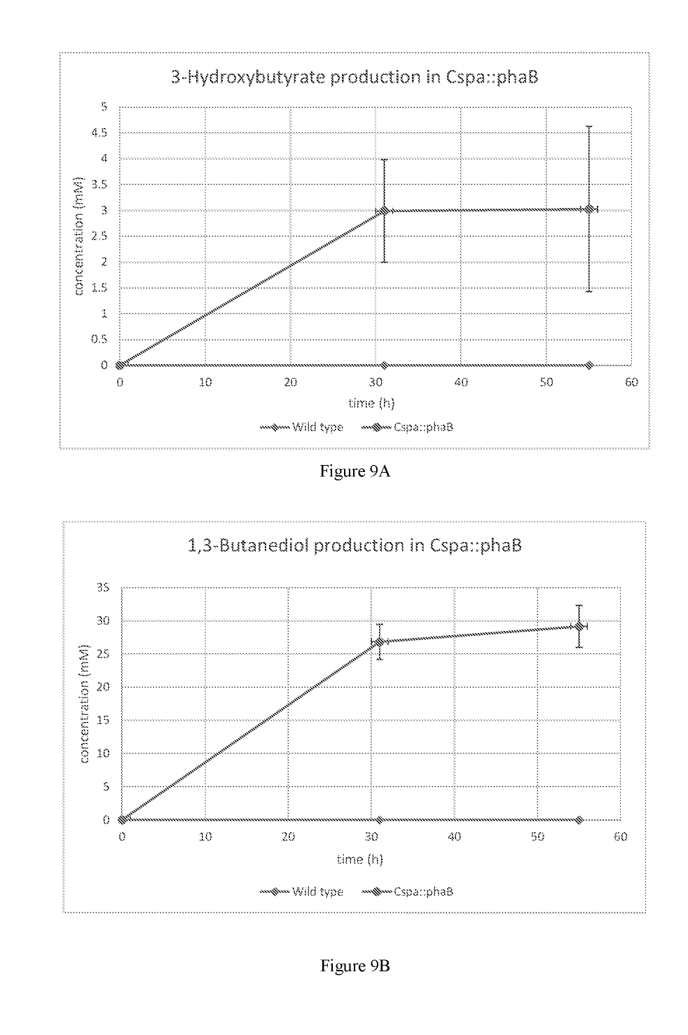

Integration of phaB into the genome of C. saccharoperbutylacetonicum leads to the production of two chiral chemicals—(R)-1,3-butanediol and (R)-3-hyrdoxybutyrate as shown in FIGS. 9A and 9B. Amounts produced were 0.3 g/L 3-hydroxybutyrate and 3.2 g/L 1,3-butanediol.

### Example 6

### C. saccharoperbutylacetonicum (Genome Integration vs Replicative Plasmid)

### 1) Gene Synthesis

The gene Cupriavidus necator PhaB was codon optimised for Clostridia. FIG. 2 shows one example of the codon optimised sequence which was synthesized by Gene Art® (Thermo Fisher Scientific).

### 2) Strain Development

In one strain PhaB was integrated into the genome of C. saccharoperbutylacetonicum using the published ACE method based on pyrE (for example as described WO2009101400). In a second strain phaB was expressed on a replicative pMTL82151 plasmid under the control of pfdx promoter. The plasmid was transformed into Cspa using standard electroporation protocol for anaerobic Clostridia.

The correct genotype of each transformant was confirmed using gene specific primers.

3) Fermentation for C. saccharoperbutylacetonicum

### Growth Method

Transformants were grown overnight as below using suitable culture media include but are not limited to FMC and CGM, as described above.

Samples for metabolic analysis were taken at regular interval and analysed for (R/S)-3-hydroxybutyrate (R/S-HB) and (R/S)-1,3-butanediol (R/S-1,3-BDO).

### Analysis

Supernatant samples were analysed using a Aminex Ion-Exclusion Column (HPX-87H, 300 mm 7.8 mm, Bio-Rad) connected to an HPLC. Metabolites were eluted with 5 mM H₂SO₄ at a flow rate of 0.5 ml min.

Chirality analysis of produced 3-hydroxybutyrate and 1,3-butanediol was carried out using HPLC-MS. The samples were derivatized using DATAN (Diacetyl-tartaric Anhydride) and separation of the S and R form was carried out using a standard non-chiral LC column (Agilent Zorbax Eclipse Plus C18, 2.1×150 mm, 1.8 um). Briefly, 10 μl supernatant was mixed with 250 μl methanol. Samples were dried down at 50° C., followed by the addition of 50 μl of freshly prepared DATAN solution (200 g/l DATAN in dichloromethane:acetic acid 4:1 (v/v). Samples were incubated for 120 min at 75° C., followed by evaporation step. Dried down samples were suspend in 500 μl water and analysed by LC-MS.

### Results

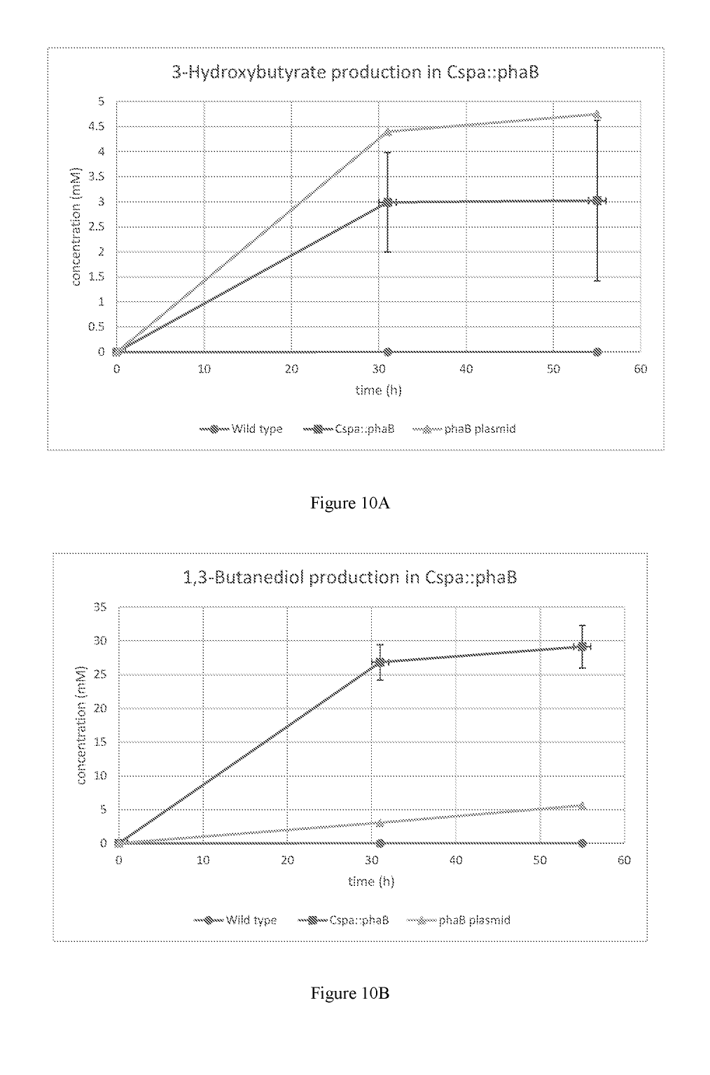

Integration of phaB into the genome of C. saccharoperbutylacetonicum and replicative plasmid expression leads to the production of two chiral chemicals—(R)-1,3-butanediol and (R)-3-hyrdoxybutyrate, as shown FIG. 10A and 10B.

Comparison of plasmid expression versus integration of phaB showed an unexpected result. Integration of phaB into the genome leads to a decrease in (R)-3-hyrdoxybutyrate titres while a 6× increase in (R)-1,3-butanediol production was observed when phaB was integrated into the genome.

As the plasmid, and subsequently phaB exist in multiple copy numbers, a greater product titre would be expected as seen for (R)-3-hyrdoxybutyrate. However this is not observed for (R)-1,3-butanediol production. Gene integration leads to increased (R)-1,3-butanediol compared to plasmid expression, indicating further regulatory mechanism and possible feedback inhibition by increased enzyme availability within the (R)-1,3-butanediol and (R)-3-hyrdoxybutyrate pathway.

1. A method of producing (R)-3-hydroxybutyric acid, and/or a salt thereof, and/or (R)-1,3-butanediol, the method comprising culturing a Clostridium species comprising a non native gene capable of expressing (R)-3-hydroxybutyryl-CoA dehydrogenase.

2. A method as claimed in claim 1 comprising culturing the Clostridium species under anaerobic or microaerophilic conditions.

3. A method as claimed in claim 1, comprising the step of purifying the produced (R)-3-hydroxybutyric acid and/or salt thereof and/or the (R)-1,3-butanediol.

4. A method as claimed in claim 1, wherein the gene is PhaB.

5. A method as claimed in claim 1, wherein:
the (R)-3-hydroxybutyric acid isomer form comprises 100% of the 3-hydroxybutyric acid formed; or
the (R)-3-hydroxybutyric acid isomer form comprises 90-100% of 3-hydroxybutyric acid formed and (S)-3-hydroxybutyric acid isomer form comprises 0-10% of the 3-hydroxybutyric acid formed.

6. A method as claimed in claim 1 wherein:
the 1,3-butanediol formed is 100% in the (R)-1,3-butanediol isomer form; or
the 1,3-butanediol formed comprises 90-100% in the (R)-1,3-butanediol isomer form and 0-10% in the (S)-1,3-butanediol isomer form.

7. A method as claimed in claim 1 wherein the Clostridium species also includes a non native gene capable of expressing thioesterase.

8. A method as claimed in claim 7, wherein the gene capable of expressing thioesterase is TesB.

9. A method as claimed in claim 1 wherein the Clostridium species comprises an S stereo specific crotonase.

10. A method as claimed in claim 1 wherein the Clostridium species comprises one or more native non substrate specific dehydrogenase/reductase enzymes able to convert (R)-3-hydroxybutyryl-CoA to (R)-3-hydroxybutyric acid and/or (R)-1, 3-butanediol.

11. A method as claimed in claim 1 wherein the Clostridium species comprises one or more non-native genes capable of expressing a non substrate specific aldehyde dehydrogenase and/or alcohol dehydrogenase able to convert (R)-3-hydroxybutyryl-CoA to (R)-1,3-butanediol.

12. A method as claimed in claim 1 wherein the non native gene capable of expressing (R)-3-hydroxybutyryl-CoA dehydrogenase is integrated into the chromosome of the Clostridium species.

13. A method as claimed in claim 1 wherein the Clostridium species comprises reduced or knocked out expression of a native gene involved in the production of butanol, acetone and ethanol, butyrate, acetate and/or lactate.

14. A method as claimed in claim 13 wherein the Clostridium species comprises reduced or knocked out expression of the native ptb, buk, hbd and/or alcohol/aldehyde dehydrogenases of the Clostridium species.

15. (R)-3-hydroxybutyric acid, and/or a salt thereof, and/or (R)-1,3-butanediol produced by a method of claim 1.

16. A Clostridium species comprising a non native gene capable of expressing (R)-3-hydroxybutyryl-CoA dehydrogenase.

17. A Clostridium species as claimed in claim 16 wherein the non-native gene is PhaB.

18. A Clostridium species as claimed in claim 16 which also includes a non native gene capable of expressing thioesterase.

19. A Clostridium species as claimed in claim 16 comprising an S stereo specific crotonase.

20. A Clostridium species as claimed in claim 16, comprising one or more native non substrate specific dehydrogenase/reductase enzymes able to convert (R)-3-hydroxybutyryl-CoA to (R)-3-hydroxybutyric acid and/or (R)-1, 3-butanediol.

21. A Clostridium species as claimed in claim 16 comprises one or more non-native genes capable of expressing a non substrate specific aldehyde dehydrogenase and/or alcohol dehydrogenase able to convert (R)-3-hydroxybutyryl-CoA to (R)-1,3-butanediol.

22. A Clostridium species as claimed in claim 16, wherein the Clostridium species is C. acetobutylicum, C. butyricum or C. saccharoperbutylacetonicum.

23. A Clostridium species as claimed in claim 16 wherein the non native gene capable of expressing (R)-3-hydroxybutyryl-CoA dehydrogenase is integrated into a chromosome of the Clostridium species.

24. A Clostridium species as claimed in claim 16, wherein the Clostridium species comprises reduced or knocked out expression of a native gene involved in the production of butanol, acetone and ethanol, butyrate, acetate and/or lactate.

25. A Clostridium species as claimed in claim 24, wherein the Clostridium species comprises reduced or knocked out expression of the native ptb, buk, hbd and alcohol/aldehyde dehydrogenases of the Clostridium species.

26. A method of producing the Clostridium as defined in claim 16, comprising incorporating a non native gene capable of expressing (R)-3-hydroxybutyryl-CoA dehydrogenase into the Clostridium species.

27. A method according to claim 26 wherein the non-native gene is integrated into a chromosome of the Clostridium species.


SEQUENCE LISTING (2 sequences)

SEQ ID NO: 1 - DNA, 744 bp, Artificial Sequence
  Codon optimised phaB gene
atgactcaaagaatagcttatgtgactggaggaatgggtggaataggaactgcaatatgccaaaggttagctaaagatggatttagagtagttgctggatgtggtccaaactctccaagaagagaaagatggcttgaacagcaaaaagctttaggttttgactttgtagcaagtgaaggaaacgttgctgactgggattcaactaagacagcttttgataaagttaaagcagaagttggagaggtagatgtattaataaataatgctggaataactagagacgttgtttttagaaaaatgacaagagcagactgggatgcagtaattgatactaatttaactagcttatttaatgtgactaagcaagtaatagacggtatggctgatagaggatggggaagaatagtaaatatatcaagtgtaaacggtcagaaaggtcaatttggacagacaaattattcaacagcaaaagctggattacatggttttactatggcattagcacaggaagttgcaacaaagggagttactgttaatactgtttctccaggatatattgcaacagatatggttaaagcaattagacaagacgttttagacaaaattgtaggaacaatacctgttaaaagacttggagagcctgaggagattgcaagtatttgcgcttggctttcatctgatgaaagtggtttttctacaggtgcagattttagtttaaatggtggtcttcacatgggttagtaa

SEQ ID NO: 2 - DNA, 861 bp, Artificial Sequence
  Codon optimised TesB gene
atgagccaggcacttaagaatcttcttacattacttaatcttgaaaaaattgaagagggtttatttagaggacaaagtgaggatctaggtttaagacaagtttttggaggacaagtagttggtcaagctttatacgctgctaaagaaacagttcctgaagaaagattagtacactcttttcactcatattttttaagaccaggtgatagcaaaaaaccaataatatatgatgtagaaactttaagagatggaaatagtttttctgctagaagagttgcagctatacaaaatggaaaacctattttttatatgacagcaagttttcaggcacctgaagcaggatttgaacatcaaaagactatgccatctgctccagctccagatggtcttccttcagagacacaaatagctcaaagtcttgcacacttgctaccaccagttttaaaagataagtttatttgtgataggccattggaagtgagaccagtggaatttcataatcctcttaaaggtcatgttgctgaaccacatagacaagtatggataagagcaaacggtagtgtaccagatgatcttagagtacatcagtatcttttaggttatgcttcagatcttaactttttgccagttgcactacagcctcatggaataggatttttagaaccaggaattcagattgcaacaatagatcattcaatgtggtttcataggccatttaatcttaatgaatggcttttatatagtgttgaatctacttctgcttctagcgctagaggatttgtaagaggagagttttatactcaagatggtgttttagtagcatcaactgttcaagaaggtgttatgagaaatcataactaa